(19) United States
(12) Patent Application Publication
(10) Pub. No.: US 2025/0321242 A1
(43) Pub. Date: Oct. 16, 2025

(54) INDICATOR IN SOLUTION AS A MEANS OF DETERMINING DOSAGE

(71) Applicant: Amano Technologies, Inc., Provo, UT (US)

(72) Inventors: Derek Bryce Shields, Provo, UT (US); Bryce Holden Jenkins, Orem, UT (US)

(21) Appl. No.: 19/049,967

(22) Filed: Feb. 10, 2025

Related U.S. Application Data
(60) Provisional application No. 63/552,024, filed on Feb. 9, 2024.

(51) Int. Cl. G01N 35/10; G01N 21/78

(52) U.S. Cl. CPC G01N 35/1002; G01N 21/78; G01N 35/1016

(57) ABSTRACT

The present invention is a system and method for real-time reagent verification using dye-based calibration in colorimetric chemical measurement systems that does not assume precise reagent addition, wherein the system actively monitors and verifies reagent dosing by incorporating a quantifiable dye into the reagent formulation, and by measuring the dye's light absorption at a fixed wavelength the system determines whether the correct reagent volume has been introduced and dynamically adjusts calibration curves to enhance measurement accuracy, wherein the system operates in real-time, utilizing an incremental dosing mechanism with automated feedback to ensure precise chemical analysis, and thereby improving the accuracy, reliability, and scalability of reagent-based chemical measurements in applications such as water quality testing, industrial chemical analysis, and laboratory diagnostics.

### BACKGROUND

Field of the Invention: The present invention relates to the field of chemical measurement systems, specifically to methods and systems for accurately dosing reagents into liquid solutions for precise chemical analyses and applies to water quality testing, industrial processes, laboratory research, utilizing advanced sensors, dye calibration, and automated data processing to ensure consistent and reliable results, thereby addressing challenges in reagent dosing accuracy, scalability, and environmental adaptability across various industries and scientific applications.

Description Of The Prior Art: The field of chemical measurement systems and reagent dosing has evolved over decades, driven by the need for precise, reliable, and efficient methods to analyze solutions. Numerous systems have been developed to measure parameters such as pH, chlorine, alkalinity, and other chemical concentrations in water quality testing, industrial processes, and laboratory research. While these existing technologies have addressed certain challenges, they also reveal limitations that the current invention seeks to overcome.

The prior art teaches various well-known methods of measurement systems. For example, spectrophotometry is a widely used technique in chemical measurement. These systems work by passing light through a sample solution and measuring the amount of light absorbed at specific wavelengths. The absorption data is then used to calculate the concentration of a target chemical in the solution. There are several advantages to spectrophotometry. Spectrophotometers provide highly precise measurements for specific chemical parameters. It is also versatile in that these systems can measure a wide range of substances by using different wavelengths and reagents. Spectrophotometers are widely used in both industrial and laboratory settings, making them a standard tool for chemical analysis.

Most spectrophotometric systems require manual addition of reagents, leading to variability in results due to human error. In addition, factors like temperature, ambient light, and water properties can impact measurement accuracy if not carefully controlled. High-quality spectrophotometers may also be expensive and may require skilled operators, limiting accessibility for smaller-scale applications.

Another testing system is colorimetric test kits. Colorimetric test kits are a simpler and more portable solution for chemical analysis. These systems involve adding a reagent to a liquid sample, which produces a color change corresponding to the concentration of the target chemical. Results are typically compared visually to a color chart or measured using a small device.

Colorimetric kits are simple to operate and require minimal technical knowledge. These kits are compact and easy to transport, making them ideal for field testing. Compared to spectrophotometers, colorimetric systems are inexpensive.

Unfortunately, results are often based on visual interpretation, which can introduce significant variability. Furthermore, these kits are typically designed for single-use applications, making them inefficient for large-scale or continuous testing. The reagent must also be added and mixed manually, increasing the chance of user error.

Furthermore, automated systems are being developed that would be even more susceptible to errors from a variety of external factors without a proper feedback system in place.

Another system for testing is a titration system. Titration systems can be manual or can automate the process of adding reagents to a solution while monitoring chemical changes. These systems are used in both industrial and research settings for precise chemical analysis.

Automated titration eliminates user error in reagent addition, ensuring consistent results. Sensors in titration systems continuously monitor chemical changes, providing immediate feedback. Titration can be applied to various chemical analyses, including pH, alkalinity, and hardness.

However, these systems require specialized equipment and trained personnel to operate. Titration systems are often designed for controlled laboratory environments and may not perform well under variable conditions. The equipment and maintenance costs are significant, limiting accessibility for smaller applications.

Despite their strengths, the prior art systems share common limitations. Manual reagent addition and visual interpretation introduce variability. Many systems are affected by temperature, light, and water properties, reducing reliability in real-world applications. Smaller systems like colorimetric kits are unsuitable for large-scale testing, while larger systems like titration devices are cost-prohibitive for small-scale use. While automated systems offer better precision, they often lack adaptability to environmental factors or portable applications.

In summary, the use of colorimeters to read chemical levels is well-established. Existing systems rely on traditional methods of collecting data and using machine learning or regression models to create measurement equations. Automated systems in the market use syringe mechanisms to add reagents but lack post-addition verification. Importantly, many systems assume that the correct reagent amount was added but lack a method to verify it.

It would therefore be an advantage over the prior art to provide an automated chemical testing system that includes reagent-dye calibration. Unlike prior art systems, the invention calibrates reagent-dye mixtures to specific wavelengths of light, ensuring minimal interference with the reagent's chemical properties while providing measurable changes in light transmission. This calibration improves accuracy and reduces the risk of user error.

It would also be an advantage to automate reagent addition, with real-time light transmission monitoring. This ensures precise control over the reagent-to-sample ratio, reducing waste and improving accuracy.

It would be a key advantage to use a modular design that allows the system to be scaled for small-scale use (e.g., portable water testing) or integrated into large industrial processes.

### BRIEF SUMMARY

The present invention is a system and method for real-time reagent verification using dye-based calibration in colorimetric chemical measurement systems that does not assume precise reagent addition, wherein the system actively monitors and verifies reagent dosing by incorporating a quantifiable dye into the reagent formulation, and by measuring the dye's light absorption at a fixed wavelength, the system determines whether the correct reagent volume has been introduced and dynamically adjusts a calibration curve or transmittance target to enhance measurement accuracy, wherein the system operates in real-time, utilizing an incremental dosing mechanism with automated feedback to ensure precise chemical analysis, and thereby improving the accuracy, reliability, and scalability of reagent-based chemical measurements in applications such as water quality testing, industrial chemical analysis, and laboratory diagnostics.

These and other embodiments of the present invention will become apparent to those skilled in the art from a consideration of the following detailed description taken in combination with the accompanying drawings.

### BRIEF DESCRIPTION OF THE SEVERAL VIEWS OF THE DRAWINGS

FIG. 1 is a flowchart describing the steps of a first embodiment of the invention.

FIG. 2 is a flowchart of the steps for preparing a reagent-dye mixture for use in the first embodiment of the invention.

FIG. 3 is a graph showing that phenol red has an absorbance peak around the 560 nm wavelength with a smaller peak at 415 nm.

FIG. 4 is a graph showing that the dye selected for this reagent primarily affects the 620 nm wavelength.

FIG. 5 is a flowchart showing the detailed steps involved in the first embodiment of the invention.

FIG. 6 is a flowchart showing the steps for calibrating the reagent/dye mixtures.

FIG. 7 is a flowchart showing the steps of using the first embodiment to measure a characteristic of a fluid.

FIG. 8 is a chart showing an example of the generated equations.

FIG. 9 is a chart showing an example of the equation being tested.

FIG. 10 is a table comparing the improvements of the first embodiment of the invention of the prior art.

### DETAILED DESCRIPTION

Reference will now be made to the drawings in which the various embodiments of the present invention will be discussed so as to enable one skilled in the art to make and use the invention. It is to be understood that the following description illustrates embodiments of the present invention and should not be viewed as narrowing the claims which follow.

This invention provides a system and method for accurately determining the volume of reagents added to liquid samples using advanced dyes, sensors, and light-based measurements. The invention addresses challenges such as human error, environmental variability, and inaccurate reagent dosing found in prior art systems. By integrating automated calibration, dye absorption principles, and real-time data analysis, the system ensures precise chemical measurements.

The key innovations lie in combining light transmission sensors with dyes that minimally interfere with reagent properties, accurately determining the volume of reagents and dyes that are added to the sample, precise calibration of the reagent and dye mixture to create equations to be used in the analysis, and providing a reliable mechanism to monitor and calculate reagent volumes while maintaining the integrity of the chemical reaction. The core principles leverage the relationship between dye absorption properties and reagent characteristics, enabling precise, non-invasive monitoring using color sensors and light transmission technology. This approach is robust against environmental factors such as temperature variations, water properties, and interfering chemicals.

The first embodiment combines light transmission technology, precise dye calibration, and automated systems to measure reagent volumes with high accuracy. The first embodiment of the invention operates in several steps.

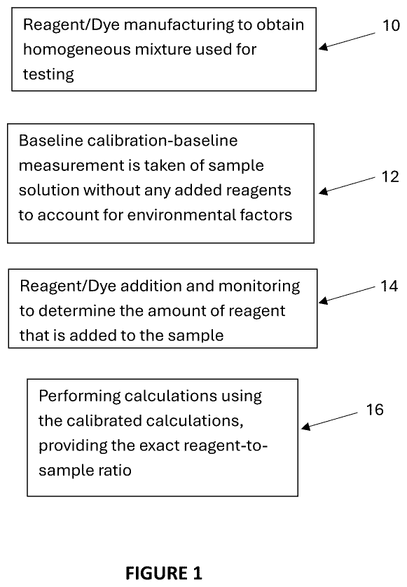

FIG. 1 shows that the first step (10) may be described as reagent/dye manufacturing to obtain a homogeneous mixture that may be used in testing

The second step (12) may be described as baseline calibration. A baseline measurement is established using the sample solution without any added reagents. This ensures that environmental factors are accounted for.

The third step (14) may be described as reagent/dye addition and monitoring. A reagent-dye mixture is added to the sample. Sensors measure changes in light transmission at specific wavelengths to determine the amount of reagent added.

The fourth step (16) is performing final calculations. The data collected is processed using calibrated equations, providing the exact reagent-to-sample ratio and ensuring accurate chemical measurements.

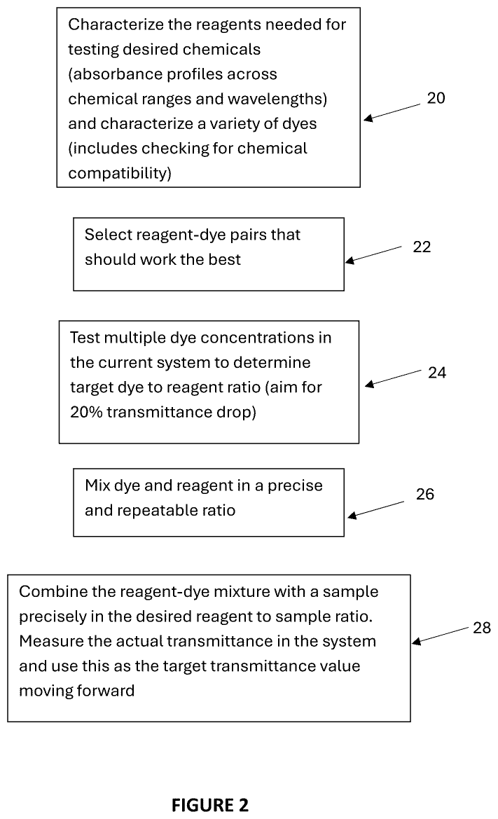

To achieve the desired measurement precision, the reagent-dye mixture is carefully prepared using the following steps as shown in FIG. 2. The first step (20) is to characterize the reagents needed for testing desired chemicals (absorbance profiles across chemical ranges and wavelengths) and characterize a variety of dyes (includes checking for chemical compatibility).

Based on the spectral properties of the reagent and target wavelength ranges, dyes are chosen for their compatibility. The second step (22) is to select reagent-dye pairs that should work the best. For example, red dyes (e.g., Red 3 and Red 40) may be used for reagents like bromocresol green. Yellow dyes may be used for thiosulfate. Yellow dyes may be used for other indicators like phenol red.

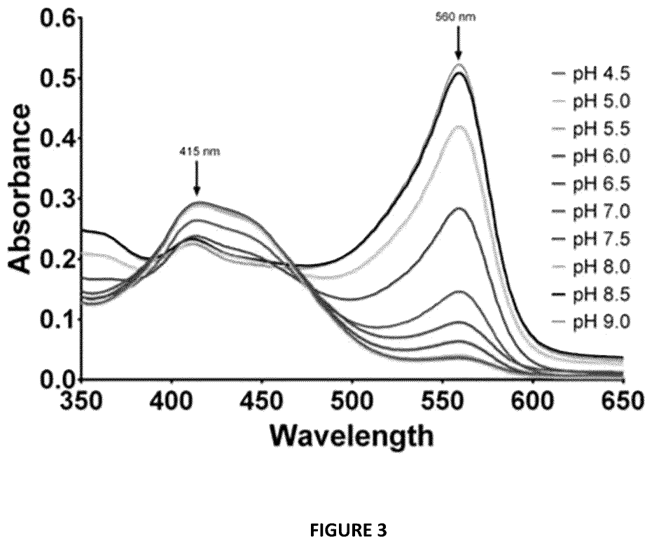

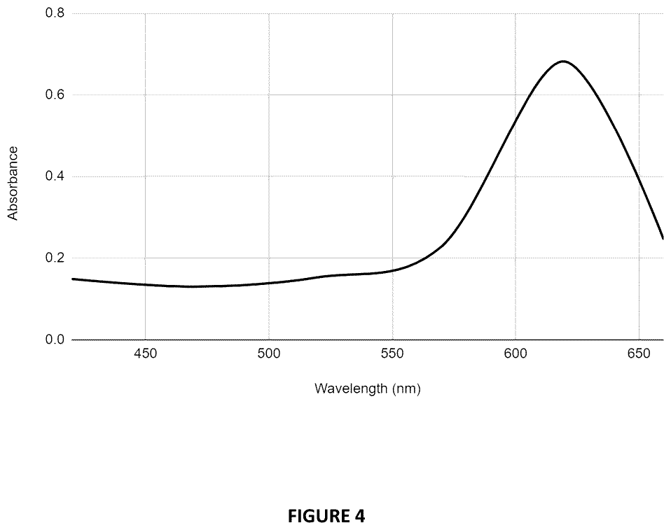

As an example regarding dyes, phenol red has an absorbance peak around the 560 nm wavelength with a smaller peak at 415 nm (see FIG. 3). Since the system measures at the 470, 520, and 620 nm wavelengths, the dye selected for this reagent primarily affects the 620 nm wavelength as shown in FIG. 4 so as not to affect the function of the phenol red. This allows the use of the 620 nm wavelength to determine whether the correct dose of reagent has been added to the sample before measuring the other wavelengths to determine the pH value in the sample.

Other factors that had to be considered when selecting dyes include the following. First, when one reagent gets used in combination with more than one additional reagent. Thiosulfate gets used with phenol red (primarily in the 520 nm wavelength) and bromocresol green indicator (primarily in the 620 nm wavelength). To avoid interfering with either, the dye selected for that reagent affects primarily the 470 nm wavelength.

A second factor is how the sample might affect the dyes. The samples may include high chlorine levels so it is important to ensure that the chlorine doesn't also affect the selected dye, which could result in false readings. Of the selected dyes, only the dye selected for thiosulfate had negative side-effects with chlorine. When tested with thiosulfate (a chlorine inhibitor) those effects became negligible. The dyes were also tested at a variety of pH and alkalinity levels for consistency.

A third factor is whether the dyes could affect the measured physical properties of the sample. The dyes selected all have neutral characteristics and are in small enough volumes so as not to impact the sample being tested.

It should be understood that these dyes are only examples and that other dyes may be substituted and that they should not be considered as limiting of the dyes that may be used.

The third step (24) is to test multiple dye concentrations in the current system to determine target dye to reagent ratio (aim for 20% transmittance drop). The reagent-dye mixture may be tested at various wavelengths (e.g., 470 nm, 560 nm, and 610 nm) to determine its absorption profile. However, testing is most likely done at the expected ideal wavelength that the dye is expected to have the greatest effect. The ideal mixture causes a 20% reduction in light transmission at the target wavelength, ensuring measurable changes without significantly altering the reagent's chemical properties. This purpose is to calibrate the relationship between the transmittance measured and the ratio of reagent-dye mixture to sample.

It should be understood that the selected wavelengths and the 20% reduction in light transmission are values that may change but are examples of possible values used in the first embodiment.

It is noted that the system relies on advanced sensors and light-emitting components to monitor light transmission. The key components include LEDs that emit light at specific wavelengths for precise measurements, photosensors that detect the amount of light transmitted through the sample solution, and then capturing data for processing.

The LED light sources emit light at specific wavelengths (e.g., red, blue, and green) to measure dye absorption. White LEDs may be used for broader-spectrum analysis.

In step four (26), dye and reagent are mixed in a precise and repeatable ratio and used in that ratio moving forward.

In step five (28) the reagent-dye mixture is combined with a sample precisely in the desired reagent to sample ratio and the actual transmittance in the system is measured. This is the target transmittance value moving forward.

Environmental sensors may be used to monitor temperature, ambient light, pressure and other variables to ensure accurate readings. These components work together to provide precise, real-time data for reagent dosing.

Experimental validation was done in controlled experiments to evaluate its accuracy, efficiency, and reliability. Multiple reagent-dye mixtures were tested across various sample solutions, including those with different pH levels, chlorine concentrations, and alkalinity.

Light transmission was measured at specific wavelengths for each reagent-dye mixture. The results were compared to baseline measurements to ensure consistent dosing.

The system demonstrated high accuracy. Reagent volumes were measured with minimal deviation from target values. Accurate readings were maintained despite changes in temperature and water properties. These results confirm the system's reliability and suitability for real-world applications.

The first embodiment may be defined as using dyes as an additional verification mechanism to determine how much reagent was added to a sample being tested. The prior art relies on trusting that the correct amount of reagent was added but cannot verify it after reagent addition to a sample. In contrast, the first embodiment actively tracks reagent addition in real-time using a dye, thereby enabling detection of under or over-dosing. Furthermore, adjustments may be made to a transmittance target to dynamically improve measurement accuracy.

The first embodiment ties a known amount of dye to the reagent volume. If 1% of the reagent contains dye, then the dye concentration directly reflects the reagent amount that was added. The first embodiment may adjust readings if too little or too much reagent was used. The first embodiment therefore eliminates reliance on fixed mechanical hardware (e.g., precise pumps) by allowing real-time verification.

The first embodiment uses incremental reagent addition with continuous monitoring. When the reagent is added to the sample, it initially adds reagent quickly (“turbo mode”) and slows down as it approaches the target level (“creep mode”). The system then checks light absorption at different wavelengths to determine: 1) when to stop reagent addition, 2) whether the reagent is fully mixed, and 3) if additional adjustments are needed. This automated approach reduces human error and improves accuracy over traditional manual or semi-automated systems dosing systems.

This dye-based approach of the first embodiment ensures that the final chemical reading is accurate, even if external factors (e.g., temperature, pressure) affect the reagent addition process. This adds flexibility in hardware choices. For example, because the system can adjust for variations in reagent delivery, expensive precision pumps are not required.

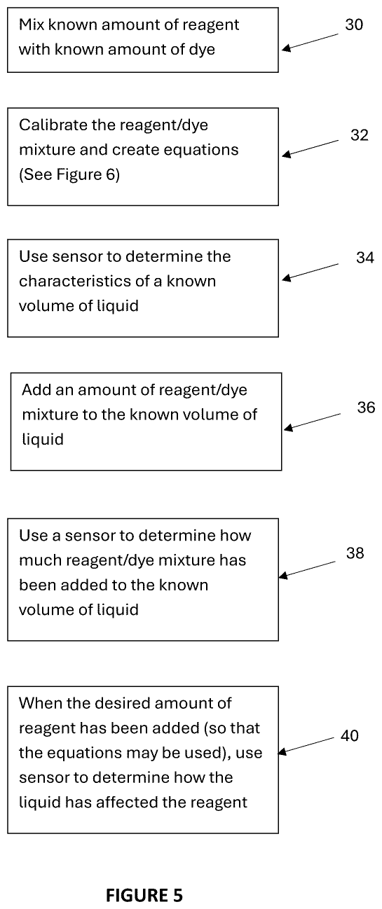

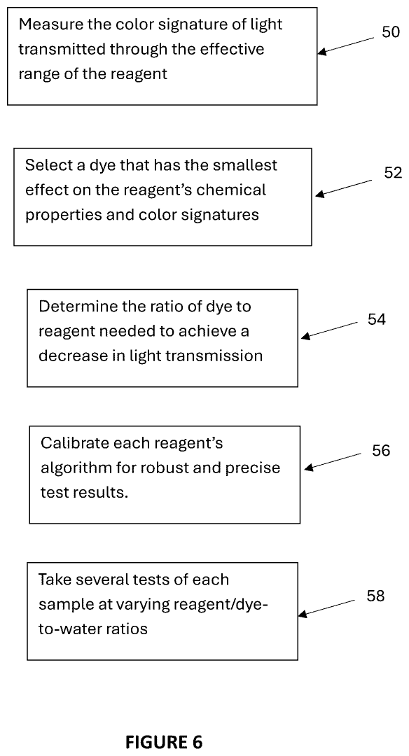

More detail on the first embodiment is given below. FIG. 5 shows the steps involved in the first embodiment of the invention. In the first step (30) a known amount of reagent is mixed with a known amount of dye. The next step (32) is to calibrate the reagent/dye mixture and create equations as shown in FIG. 6. The next step (34) is to use the sensors to determine the characteristics of a known volume of liquid. The following step (36) adds an amount of reagent/dye mixture to the known volume of liquid. The next step (38) uses a sensor to determine how much reagent/dye mixture has been added to the known volume of liquid. Lastly in step (40) when the desired amount of reagent has been added (so that the equations may be used), use sensors to determine how the liquid has affected the reagent.

In FIG. 6 the first step (50) of calibrating the reagent/dye mixtures is performed by first using sensors from the first embodiment or a similar device to measure the color signature of light transmitted through the effective range of the reagent.

With that range of information, a dye is selected in step 2 (52) that has the smallest effect on the reagent's chemical properties and color signatures.

The next step 3 (54) is to determine the ratio of dye to reagent needed to achieve a decrease in light transmission. In the first embodiment, the decrease may be selected to be a 20% decrease. The 20% value may be adjusted as needed and should not be considered a limiting factor of the first embodiment. This is dependent on the color of the dye, the color of the reagent, and the thickness of the volume of water being measured. The ideal values are identified by testing different concentrations of dye in water in specifically sized containers.

The next step 4 (56) is to calibrate each reagent's algorithm for robust and precise test results. This may be accomplished by preparing several samples of water per chemical type (PH, Alkalinity, Chlorine, etc.). These samples cover the appropriate testing range of the chemicals as well as varying external related conditions such as temperature, or other chemical levels.

The next step 5 (58) is to take several tests of each sample at varying reagent/dye-to-water ratios. Each water sample has a reagent/dye mixture added to it and is mixed thoroughly. Using LEDs and photosensors, transmitted light values at a range of wavelengths are collected and stored. Those values along with their accompanying water sample characteristics are used to create large multivariable equations. The calibration tests are rerun as new conditions are discovered.

Once the reagent and dye mixture are calibrated, the reagent and dye mixture may be manufactured in larger volumes. This may be accomplished by acquiring a container of reagent and determining the volume.

Using the given volume of reagent, determine what volume of dye needs to be added. A matrix may be created that has ideal dyes picked out for each reagent as well as ideal reagent-to-dye concentrations to allow the best ratio of readability to reagent concentration.

The next step is to add the determined volume of dye to the reagent. The reagent/dye solution is mixed until it is homogeneous and then set aside for use in production.

When the reagent/dye solution is shipped to a user, the equations are sent with it. For example, a given reagent/dye solution may be assigned to a bar code, and an associated set of equations are then transmitted to a device that uses the reagent/dye solution.

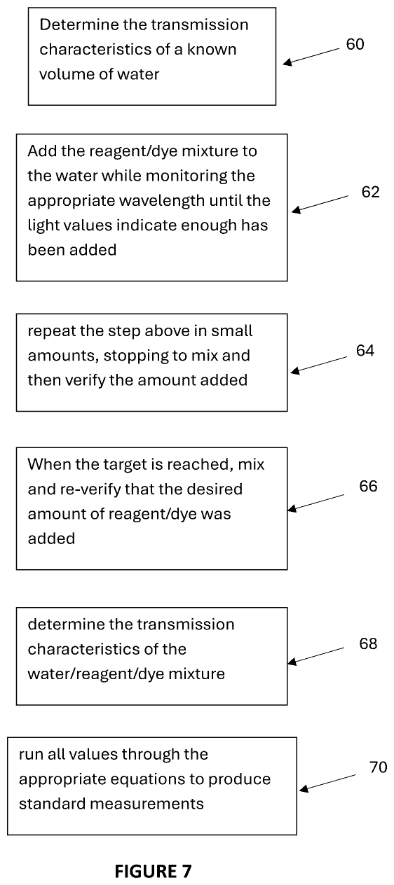

FIG. 7 shows that the first step (60) in using the reagent/dye solution begins by determining the transmission characteristics of a known volume of water. This step (60) may be referred to as zeroing and is used to determine the color signature of the water before the reagent/dye mixture is added.

The next step (62) is to add the reagent/dye mixture to the water while monitoring the appropriate wavelength until the light values indicate enough has been added.

The next step (64) is to repeat the step above in small amounts, stopping to mix and then verify the amount added.

Once the target is reached, the next step (66) is to mix and re-verify that the desired amount of reagent/dye was added.

Using LEDs and photosensors, the next step 68 is to determine the transmission characteristics of the water/reagent/dye mixture.

The final step (70) is to run all values through the appropriate equations to produce standard measurements. These are measurements being made for any characteristics of water that may be tested.

When testing water, different physical, chemical, and biological parameters are measured to determine its safety, cleanliness, and suitability for various uses (drinking, industrial, environmental, or agricultural purposes). Below are the key things typically measured in water testing. These characteristics may include but should not be considered as limited to physical properties, chemical parameters, biological contaminants, and radiological contaminants.

For example, chemical parameters may include chemical indicators such as pH, total dissolved solids (TDS), nutrients & organics, nitrate, nitrite, phosphate, ammonia, hardness & alkalinity, heavy metals (toxic contaminants), chlorine, chloramines, sulfates, and chlorides.

For the purposes of providing an example of the first embodiment of the invention, 1 mL of Red Dye (red 3&40) is added to 119 mL of Alkalinity indicator.

An example of data collected is for two reagents added with one zeroing step at the beginning. The zeroing measurements are 610 nm: 2292 u, 560 nm: 7528 u, 470 nm: 8410 u, White light: 13541 u, temperature: 72 F.

Thiosulfate/dye added generated the following measurements of 610 nm: 2231 u, 560 nm: 7233 u, 470 nm: 6728 u, White light: 12024 u, Temperature: 72 F. Relative transmittance is measured to be 610 nm: 97.34%, 560 nm: 96.08%, 470 nm: 80.00%. Alkalinity Indicator/dye added generated the following values, 610 nm: 1546, 60 nm: 4783, 470 nm: 4387, White light: 8093, Temperature: 72 F.

Relative Transmittance is measured at 610 nm: 69.30%, 560 nm: 66.13%, 470 nm: 65.21%.

During calibration of the reagent/dye mixture, the following equation was generated for Alkalinity. Alkalinity=({470 nm}\hat{ }2*C9)+({560 nm}\hat{ }2*C8)+({610 nm}\hat{ }2*C7)+({560 nm}*{470 nm}*c6)+({610 nm}*{470 nm}*C5)+({610 nm}*{560 nm}*C4)+({470 nm}*C3)+({560 nm}*C2)+({610 nm} *C1)+C0, where C0-C9 are coefficients. This is an example format, and any number of parameters and accompanying coefficients could be added and utilized. The alkalinity value was 131.

It is noted that two dyes were used in this example. The Thiosulfate was combined with a dye that affects the blue wavelengths. The alkalinity indicator was combined with a dye that affects the green wavelengths. Wavelengths tested are 470 nm, 560 nm, 590 nm (white light), and 610 nm.

Reagents used include Phenol red, Thiosulfate, Sodium Iodine, DPD, Lithium maleate, and an Alkalinity Indicator. Dyes used include Dye (red 40 & red 3), Blue Dye (blue 1 & red 40), Yellow Dye (yellow 5 & red 40).

Sensors and components used include a Red LED (631 nm), a Green LED (525 nm), a Blue LED (470 nm), and a White LED, a Thermistor and a Color sensor.

Possible future reagents include any liquid reagent or reagent that can be dissolved into a liquid to react with a liquid based solution. A list of reagents includes but should not be considered as limited to calcium hardness indicators, copper indicators, bromine indicators, acid demand indicators, base demand indicators, chromate indicators, starch indicators, calcium indicators, iron indicators, conductivity indicators, algae indicators, oxygen indicators, zinc indicators, nitrogen indicators, turbidity indicators, bacterial indicators, etc.

Possible dyes that may be used include but should not be considered as limited to any liquid based dye or dye that can be dissolved in a liquid. These include any dye which affects light transmittance, light reflectance, light absorbance, UV range, Infrared range, and X-rays.

Additives may also be used. This includes any additive that can be observed by a digital or analog sensor including electromagnetic, thermal, conductive, temperature, sound absorbance, sound reflectance, and viscosity. All dyes must not affect the chemistry of the reagent they are being added to either primarily or later in a test with multiple reagents.

Possible sensors including light, heat, conductivity, sound, movement, and pressure.

All of the information above may be used in an automated or manual water testing system.

In gathering data for the first embodiment of the invention, nine devices were set up to run tests on demand and produce raw light value outputs as opposed to chemical level values. The next step was to mix together a 1-gallon container of water 0 PPM Alkalinity and reasonable/safe chemical levels for all other standard pool water chemicals.

The next step was to verify the PPM of Alkalinity using 2 or more methods, specifically in this data we used a manual comparator method and a digital colorimeter method.

Each device was flushed with the new water sample and made sure there were no bubbles in the testing water sample or tubes. Then each device was run to record the transmittance data it produces including the zero values for each light and the value after reagent is added for both sensor sets. These were Red0, Green0, Blue0, White0, Red, Green1, Blue1, White1, Red0, Green0, Blue0, White0, Red1, Green1, Blue1, and White1.

The next step was to calculate the change in transmittance values r1, g1, b1, r2, g2, and b2. The next step was to average the values from sensor 1 and 2 as long as they are within range of each other, or 0.15 in this instance to obtain RT, GT, GT.

These steps were repeated with many different chemical values. In this case, the following values were used: 0, 30, 70, 100, 130, 170, 200, and 235.

When processing the data, it was manually looked through for other errors, notes and inconsistencies. Data points were flagged and removed from the data set if an error has occurred that should not occur in normal testing. The main way this was accomplished in this test was by graphing the raw values and identifying any outliers in that graph. Anything deemed an outlier is removed from the data set along with the other values associated with that test and sometimes even that device's group of tests.

All “valid” data was then plugged into a regression calculator and using different combinations of variables and relationships between those variables several equations were created.

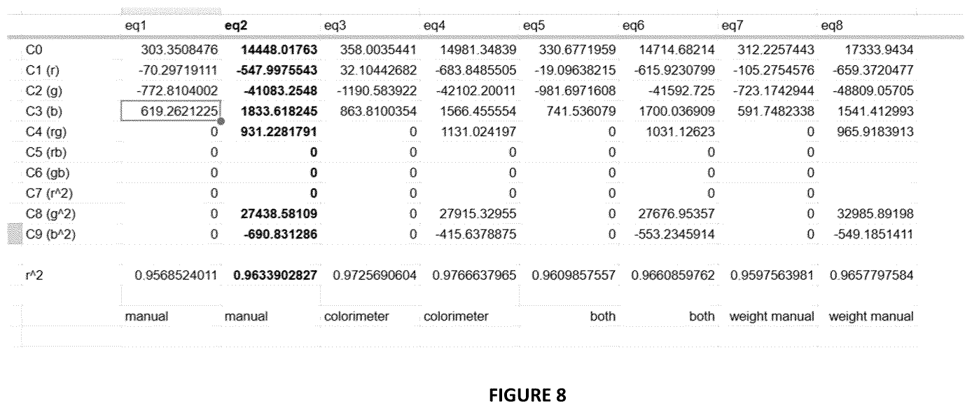

The values C0 through C9 represent the coefficients calculated given the combination of variables and data used to generate the equation. An example of the generated equations is shown in FIG. 8.

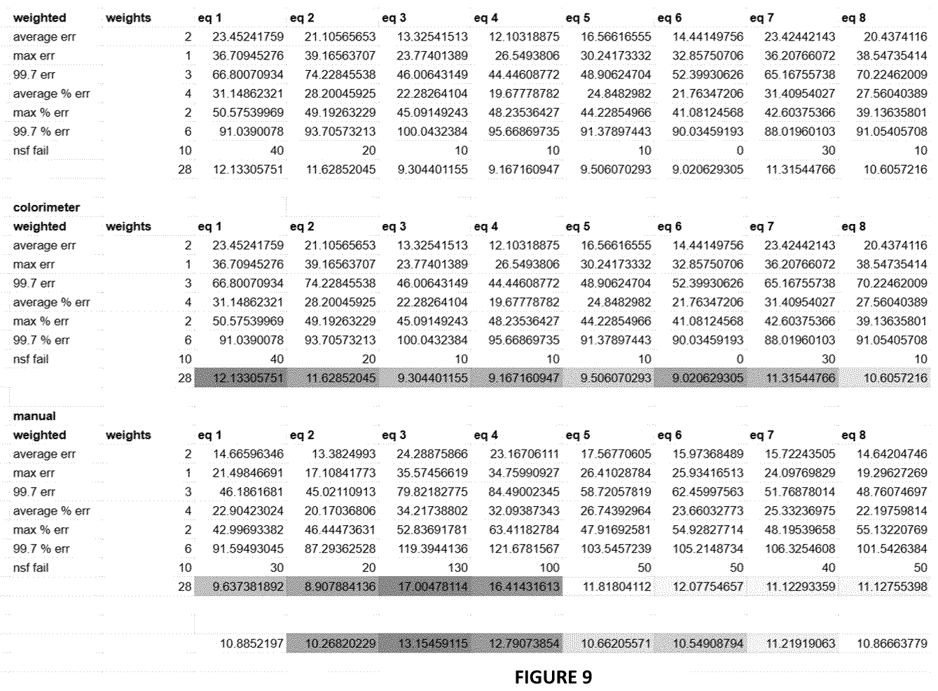

Once equations have been generated, each equation can be tested against new data to verify how accurate it will be. Accuracy values are compared in many ways including Error from one initial method, Error from both initial methods, error from the average of the initial methods, how often the calculated values stray from initial values, average error, maximum error, and cumulative error. More values can be used but the seven listed in FIG. 9 were sufficient for this example.

Using those values the best fitting equation is used. By determining which equation best fits the data without overfitting the data. As new data is collected, the equations are checked against that data and then former validation data can be added to the data set updating the equations.

It is crucial that the set of data used for validating the equations is not also being used in the creation of the equations. This equation is then written in a simplified form and added to the reagent testing profile through software and firmware.

Example: Alkalinity PPM=(RT\hat{ }2*C9)+(GT\hat{ }2*C8)+(BT\hat{ }2*C7)+(GT*BT*C6)+(BT*RT*C5)+(BT*GT*C4)+(RT*C3)+(GT*C2)+(BT*C1)+C0

This process can be done for any reagent or dye in any combination.

In summary, all colorimeter testing appears to be dependent on the quality of some initial calibration data that can be used to represent future un-calibrated/independent data. The main difference is that adding the variables created by the concentration of dye in the reagent allows the first embodiment to determine with extreme precision how much reagent has been added independently of the insertion methods accuracy or repeatability.

The accuracy of the amount of reagent being added to each test was only as accurate as the method of dispensing the reagent. Examples include droppers, syringes, vials, scales, and pumps. The first embodiment has reduced and nearly eliminated the need for the system that dispenses reagents to do so repeatably or precisely. By adding a precise amount of dye to the reagents in controlled environments, all that is needed are sensors that can precisely measure the amount of that dye in a sample. At this time, a photosensor is being used, but nearly any sensor could be used with the right additive.

Using the precise values of how much dye is in each reagent, the first embodiment can use much less complicated and expensive equipment to produce values that can be compared to the costly and precise calibration data.

The dye enables the first embodiment to calculate the quantity of reagent in the mixture while the effects of the reagent are being calculated. With no possible time delay between reagent volume calculation and reagent effect calculation, there can be no change in variables that affect the amount of reagent in a test relative to its effect.

Because the calibration data is continuously being updated, the equations and variables used in those equations could be continuously changing. While the current equations may have three variables and ten coefficients, past and future equations may have up to an infinite number of variables and coefficients. As more variables are tracked, like water temperature, time of day, and other chemical concentrations, the equations and how those equations are attained will change. The reliability of tests will only improve as additional variables are correctly incorporated.

This method pertains to the collection of additional variables, specifically those related to the dye and that it is irrelevant how data is collected, how/if equations are created, and what those equations look like. This first embodiment enables the processing of all data that could be collected using the addition of dye or additive to verify the quantity of some liquid being added to another.

Alternatives to visible dyes could include UV or IR absorbing additives. These dyes could reduce the interference of visible dyes in a test where the reagents affect visible wavelengths. The dyes would also simplify the process of addition since all reagents could use the same dye but would require new hardware that would emit and sense wavelengths in the UV or IR spectrum, respectively.

Another alternative would be combining an additive directly with another substance, either a liquid or solid, so as to “measure” the amount of that substance in a dosing application. One example is the addition of a dye powder in granular chlorine to be used in an automated dispenser. The dye would allow for accurate measuring of the chlorine concentration once dissolved.

While the added indicator can be used to determine the correct dose, the additional metric associated with the volume of reagent can be used to further increase the accuracy of the test results by adding another dimension to the compared dataset. Small differences in the volume, which would normally add to the overall error of the measurement, may be negated through larger datasets.

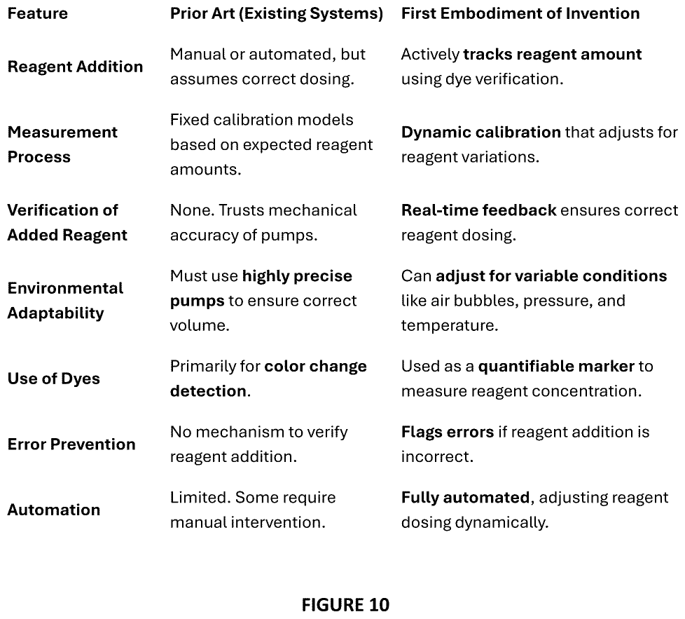

FIG. 10 is a chart showing the features of the first embodiment compared to the prior art.

In summary, the first embodiment of the invention is a method for determining and verifying reagent dosing in a liquid sample, the method comprising the steps of: 1) preparing a reagent-dye mixture by incorporating a predetermined concentration of a dye into a reagent solution, wherein the dye is selected to exhibit a measurable optical absorption property at a predefined wavelength, 2) directing a light source at the liquid sample and measuring the transmitted or absorbed light intensity at the predefined wavelength using a photosensor to determine a baseline optical property of the sample, 3) introducing a defined volume of the reagent-dye mixture into the liquid sample, 4) continuously or incrementally adding additional volumes of the reagent-dye mixture to the liquid sample, 5) monitoring real-time changes in the optical absorption or transmission of the liquid sample at the predefined wavelength to determine the actual reagent concentration in the sample, 6) comparing the detected optical changes to an expected transmittance target to verify whether the correct amount of reagent has been introduced into the sample, 7) adjusting reagent dosing in response to detected deviations from the expected optical properties, thereby ensuring precise reagent delivery and accurate chemical measurements, and 8) measuring the reagent wavelengths to generate a measured chemical value.

A summary of the hardware requirements of the first embodiment include A system for determining and verifying reagent dosing in a liquid sample, wherein the system is comprised of 1) a reagent-dye mixture, wherein the reagent is formulated with a predetermined concentration of a dye that exhibits a measurable optical absorption property at a predefined wavelength, 2) a reagent delivery mechanism configured to introduce a controlled volume of the reagent-dye mixture into the liquid sample, 3) a light source configured to emit light at the predefined wavelength to interact with the liquid sample containing the reagent-dye mixture, 4) a photosensor (color sensor) configured to detect light transmission or absorption at the predefined wavelength and generate measurement signals corresponding to the reagent concentration in the sample, and 5) a processing unit operatively connected to the photosensor, the processing unit being configured to: establish a baseline optical property of the sample before reagent addition, monitor real-time changes in optical absorption or transmission during reagent introduction, compare detected optical properties to a pre-stored transmittance target and determine whether the correct amount of reagent has been introduced into the sample based on deviations from expected optical properties.

The hardware components may also include a feedback control system configured to adjust reagent dosing in response to detected deviations from expected optical values, ensuring accurate reagent delivery, and 3) a user interface configured to display reagent concentration data, calibration adjustments, and alerts regarding potential dosing errors.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. It is the express intention of the applicant not to invoke 35 U.S.C. § 112, paragraph 6 for any limitations of any of the claims herein, except for those in which the claim expressly uses the words ‘means for’ together with an associated function.

What is claimed is:

1. A method for determining and verifying reagent dosing in a liquid sample, the method comprising:
preparing a reagent-dye mixture by incorporating a predetermined concentration of a dye into a reagent solution, wherein the dye is selected to exhibit a measurable optical absorption property at a predefined wavelength;
directing a light source at the liquid sample and measuring the transmitted or absorbed light intensity at the predefined wavelength using a color sensor to determine a baseline optical property of the sample;
introducing a defined volume of the reagent-dye mixture into the liquid sample;
continuously or incrementally adding additional volumes of the reagent-dye mixture to the liquid sample;
monitoring real-time changes in the optical absorption or transmission of the liquid sample at the predefined wavelength to determine the actual reagent concentration in the sample;
comparing the detected optical changes to an expected transmittance target to verify whether the correct amount of reagent has been introduced into the sample; and
adjusting reagent dosing in response to detected deviations from the expected optical properties, thereby ensuring precise reagent delivery and accurate chemical measurements.

2. The method as defined in claim 1 wherein the method further comprises selecting the dye in the reagent-dye mixture from a group consisting of food-grade dyes, organic dyes, and metal-based indicator dyes, chosen to minimize interference with the reagent's chemical properties.

3. The method as defined in claim 1 wherein the method further comprises selecting the predefined wavelength at which the dye exhibits optical absorption from a range between 400 nm and 700 nm to optimize detection sensitivity.

4. The method as defined in claim 1 wherein the method further comprises calibrating the optical measurement system by establishing a reference optical absorption value using a control liquid sample prior to reagent introduction.

5. The method as defined in claim 1 wherein the method further comprises pre-mixing the reagent-dye mixture in a standardized ratio to maintain a fixed dye-to-reagent proportion across multiple tests for consistency.

6. The method as defined in claim 1 wherein the method further comprises dynamically adjusting reagent dosing based on real-time optical feedback, wherein deviations in detected optical absorption values trigger automated modifications to reagent addition.

7. The method as defined in claim 1 wherein the method further comprises:
emitting light from a light emitting diode (LED) at the predefined wavelength;
measuring changes in light intensity passing through or reflected from the sample using a photosensor.

8. The method as defined in claim 1 wherein the method further comprises storing the reagent-dye mixture in a sealed, light-protected container to prevent photodegradation of the dye prior to use.

9. The method as defined in claim 1 wherein the method further comprises providing an alert or notification if the detected reagent concentration deviates beyond a predetermined threshold, indicating possible errors in reagent addition or contamination.

10. The method as defined in claim 1 wherein the method further comprises generating a transmittance target used for comparison that is dynamically updated based on historical reagent-dye mixture behavior to improve accuracy across different environmental conditions.

11. The method as defined in claim 1 wherein the method further comprises incorporating a secondary validation measurement using an alternative wavelength to cross-verify reagent concentration accuracy.

12. The method as defined in claim 1 wherein the method further comprises continuously mixing the liquid sample during reagent addition to ensure uniform distribution of the reagent-dye mixture and to reduce measurement inconsistencies.

13. The method as defined in claim 1 wherein the method further comprises using the reagent-dye mixture in applications including but not limited to water quality testing, industrial chemical analysis, biological fluid analysis, and environmental monitoring.

14. The method as defined in claim 1 wherein the method further comprises compensating for variations in ambient light conditions by incorporating real-time background light correction in the optical measurement system.

15. The method as defined in claim 1 wherein the method further comprises formulating the reagent-dye mixture to be compatible with multiple liquid sample types, including aqueous solutions, organic solvents, and biological fluids.

16. A system for determining and verifying reagent dosing in a liquid sample, wherein the system is comprised of:
a reagent-dye mixture, wherein the reagent is formulated with a predetermined concentration of a dye that exhibits a measurable optical absorption property at a predefined wavelength;
a reagent delivery mechanism configured to introduce a controlled volume of the reagent-dye mixture into the liquid sample;
a light source configured to emit light at the predefined wavelength to interact with the liquid sample containing the reagent-dye mixture;
a photosensor configured to detect light transmission or absorption at the predefined wavelength and generate measurement signals corresponding to the reagent concentration in the sample; and
a processing unit operatively connected to the photosensor, the processing unit being configured to: establish a baseline optical property of the sample before reagent addition, monitor real-time changes in optical absorption or transmission during reagent introduction, compare detected optical properties to a pre-stored transmittance target, and determine whether the correct amount of reagent has been introduced into the sample based on deviations from expected optical properties.

17. The system as defined in claim 16 wherein the system is further comprised of:
a feedback control system configured to adjust reagent dosing in response to detected deviations from expected optical values, ensuring accurate reagent delivery; and
a user interface configured to display reagent concentration data, calibration adjustments, and alerts regarding potential dosing errors.

18. The system as defined in claim 16 wherein the system is further comprised of a reagent delivery mechanism including an automated pump or syringe system configured to dispense precise incremental volumes of the reagent-dye mixture into the liquid sample based on real-time feedback from the processing unit.

19. The system as defined in claim 16 wherein the system is further comprised of a light source including one or more LEDs emitting at multiple predefined wavelengths, and the photosensor is configured to measure optical absorption across multiple wavelengths to enhance reagent concentration verification accuracy.

20. The system as defined in claim 16 wherein the system is further comprised of a processing unit that is further configured to dynamically adjust the transmittance target based on environmental factors, including ambient light conditions, temperature variations, and previous measurement trends, to improve accuracy over time.
